(19) United States
(12) Patent Application Publication
(10) Pub. No.: US 2014/0031781 A1
(43) Pub. Date: Jan. 30, 2014

(54) PRESSURE APPLICATION FOR HEMOSTATIS

(71) Applicant: Lydda Razon-Domingo, Des Plaines, IL (US)

(72) Inventor: Lydda Razon-Domingo, Des Plaines, IL (US)

(21) Appl. No.: 13/939,602

(22) Filed: Jul. 11, 2013

Related U.S. Application Data
(60) Provisional application No. 61/741,085, filed on Jul. 12, 2012.

(51) Int. Cl. A61F 13/56; A61B 17/132

(52) U.S. Cl. CPC A61F 13/56; A61B 17/1325
USPC 604/385.03

(57) ABSTRACT

Coaguband is a device for applying direct pressure on a bleeding site. It has an adhesive band, and an absorbent in the center which encloses a dome-shaped rubber or plastic component. The band anchors the device on the bleeding site at the same time applying pressure on it, while the dome-shaped component multiplies the pressure exerted by the band. The absorbent absorbs unclotted blood. In most cases, with its dome-shaped component multiplying the pressure exerted by its adhesive band, it effectively replaces the human thumb in applying direct pressure on a bleeding site, freeing the medical personnel for other duties.

### CROSS REFERENCE TO RELATED APPLICATIONS

This application claims the priority of U.S. patent application Ser. No. 61/741,058 filed on Jul. 11, 2012, Inventor Lydda R. Domingo, entitled “Pressure Application for Hemostatis.” The entire disclosure of this aforementioned provisional patent application is hereby incorporated in its entirety by reference thereto.

### FIELD OF THE INVENTION

The invention is under the field of hemostatic products use for direct pressure to stop external bleeding.

### BACKGROUND OF THE INVENTION

To stop external bleeding from relatively superficial causes, 4×4s (gauze) are generally use to apply direct pressure to the bleeding site. This is usually adequate. However, in cases where there is high pressure (like bleeding from an artery) or an anticoagulant was administered during a medical procedure or therapy (e.g. hemodialysis and cardiac catheterization), the bleeding is prolonged and applying direct pressure using 4×4s (gauze) becomes inefficient.

In hemodialysis, a large vein (arteriovenous (AV) fistula) or artificial vessel (arteriovenous (AV) graft) is joined to an artery. These become an access site where blood is drawn—to be filtered through the dialysis machine—and returned. Being joined to an artery, the AV fistula and AV graft have high pressure. During the treatment, blood thinner is administered to prevent clotting of blood in the dialysis lines. After the treatment, the large bore dialysis needles are removed and direct pressure using 4×4s (gauze) is applied to the area. Because of high pressure in the access site and blood thinner administered during treatment, bleeding is usually prolonged. This results to a lot of 4×4s (gauze) used, a lot of blood loss, and a lot of time spent—both the personnel's and the patient's. Layers of 4×4 (gauze) put on to the bleeding site make it difficult to apply effective pressure. Constant changing of the 4×4s (gauze) also contributes to prolonged bleeding.

The human factor of inconsistent application of pressure adds to the problem of prolonged bleeding in dialysis, aggravated by the fact that an access site may also move from side to side and thus reducing the pressure applied.

### SUMMARY OF THE INVENTION

From the foregoing, it is seen that it is a problem in the art to provide a device meeting the above requirements. According to the present invention, a device and process are provided which meets the aforementioned requirements and needs in the prior art.

Specifically, the device according to the present invention is designed for consistent and effective pressure applied to the bleeding site, eliminating the human factor. It also stabilizes a movable hemodiaysis access. In most cases, with the design that included an adhesive band, it can be applied and left alone freeing the medical personnel to do other things. Generally, it speeds up stoppage of bleeding.

The invention is a device designed to stop bleeding from various sources, particularly the following:
Access sites for Hemodialysis: Fistula and Graft.
Internal jugular or femoral catheter site.
Cardiac Catheterization sites.
Phlebotomy procedure: Arterial or Venous puncture for laboratory analysis.
Intravenous injection sites
Wounds from various causes

Other objects and advantages of the present invention will be more readily apparent from the following detailed description when read in conjunction with the accompanying drawings.

### BRIEF DESCRIPTION OF THE DRAWINGS

FIG. 1 is a schematic perspective view of the coaguband according to the present invention.

FIG. 2 is an exploded view schematically showing the assembly of the parts forming the coaguband of FIG. 1 showing a strip portion, a dome-shaped portion, and an absorbent portion.

FIG. 3 is a sectional view taken along the center of the dome-shaped portion shown in FIGS. 1 and 1, taken transversely to a longitudinal direction of the strip portion.

FIG. 4 is a side elevational view of a truncated dome-shaped portion, for use with the embodiment of FIGS. 1-3 and with the embodiment of FIG. 6.

FIG. 5 is a side elevational view of another variation of a truncated dome-shaped portion, for use with the embodiment of FIGS. 1-3 and with the embodiment of FIG. 6.

FIG. 6 is a side sectional view of another embodiment of a pressure applying portion having an absorbent and coagulant, in the coaguband of the present invention.

FIG. 7 is a top elevational view of the embodiment of FIG. 6, according to the present invention.

### DETAILED DESCRIPTION OF THE INVENTION

FIG. 1 is a schematic perspective view of a coaguband 100, having a strip portion 10 having adhesive on its lower surface so as to form an adhesive surface. Extending below the strip portion 10 and in dashed outline showing a dome-shaped portion 30 and an absorbent portion 20.

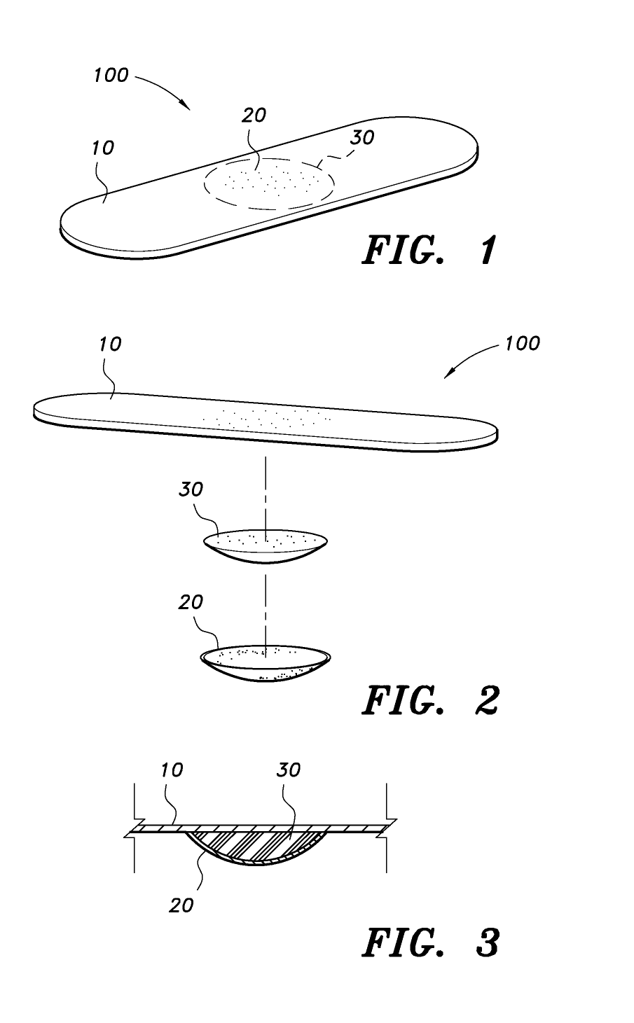

The dome-shaped portion 30 and the absorbent portion 20 are shown in further detail in FIGS. 2 and 3, as described further hereunder.

In use, the strip portion 10 is applied over a desired location with the adhesive surface facing down so that the adhesive surface can contact surrounding tissue so as to secure the coaguband 100 to the tissue over the desired location, and so that the dome-shaped portion 30 is urged downwardly against the desired location so as to force the absorbent portion 20 directly into contact with the desired location.

FIG. 2 is an exploded view schematically showing the assembly of the parts forming the coaguband 100 of FIG. 1 showing the strip portion 10, the dome-shaped portion 30, and the absorbent portion 20. The adhesive layer is indicated along the bottom surface of the strip portion 10 as viewed in FIG. 2.

FIG. 3 is a sectional view taken along the center of the dome-shaped portion 30 shown in FIGS. 1 and 1, taken transversely to a longitudinal direction of the strip portion 10. The invention is discussed in further detail below.

The Coaguband 100 is a device for applying direct pressure on a bleeding site. It has an adhesive band 10, an absorbent portion 20 having in its center the dome-shaped rubber or plastic component 20. The band 10 anchors the device 100 on the bleeding site at the same time applying pressure on it, while the dome-shaped component 30 multiplies the pressure exerted by the band 10. The absorbent portion 20 absorbs unclotted blood. The coaguband 100 design makes for a more efficient device for stopping bleeding than the traditional method of a medical personnel applying pressure with 4×4 (gauze). In most cases, with its dome-shaped component 30 multiplying the pressure exerted by its adhesive band 10, it effectively replaces the human thumb in applying direct pressure on a bleeding site, freeing the medical personnel for other duties.

The coaguband 100 is a hemostatic device which has a dome-shaped rubber or plastic component 30 that is enclosed in an absorbent portion 20. The sizes vary for the intended usage, as follows:
Small—for intravenous and phlebotomy procedure all of which are fastened to an adhesive band.
Medium—for small to medium bleeders in hemodialysis, internal jugular or femoral catheter, and cardiac catheterization.
Larger—for heavy bleeders in hemodialysis, internal jugular or femoral catheter, and cardiac catheterization bleeding.
Extra large—for heavy bleeding wounds of various causes.

A further embodiment of the invention will have the hemostatic device 100 fastened to an adhesive band which also differs in size—both in width and length—and in the nature of components.

At the center of the adhesive band 10 is a dome-shaped plastic or rubber component 30, which is enclosed by the absorbent portion 20. In FIG. 2, the absorbent portion is shown as a hollow shell-shaped portion capable of receiving the dome-shaped portion 30.

The adhesive portion 10 forms a band, and anchors the coaguband 100 on the bleeding site. This adhesive portion 10 can be relatively short, comparable to a regular size “Band Aid”, or this can be relatively long and capable of wrapping around a patient's arm. The short version of the adhesive portion 10 can be used for light to medium bleeding. The longer version of the adhesive portion can apply more pressure and can be used for medium to heavy bleeding.

The dome-shaped rubber or plastic component 30 multiplies the pressure applied on the bleeding site by the adhesive. This also stabilizes a movable dialysis access site (fistula or graft), preventing it from moving out of the point of pressure.

The absorbent portion 20 absorbs the blood efficiently, and can be composed of any known type of absorbent material known in the medical or dental arts for use with bleeding.

Advantages:
Effective pressure to lessen the time of bleeding thereby prevents unnecessary blood loss.
Convenient and efficient way to stop external bleeding.
The user is able to control the pressure applied to the site and direct the pressure to the center of the bleeding site.

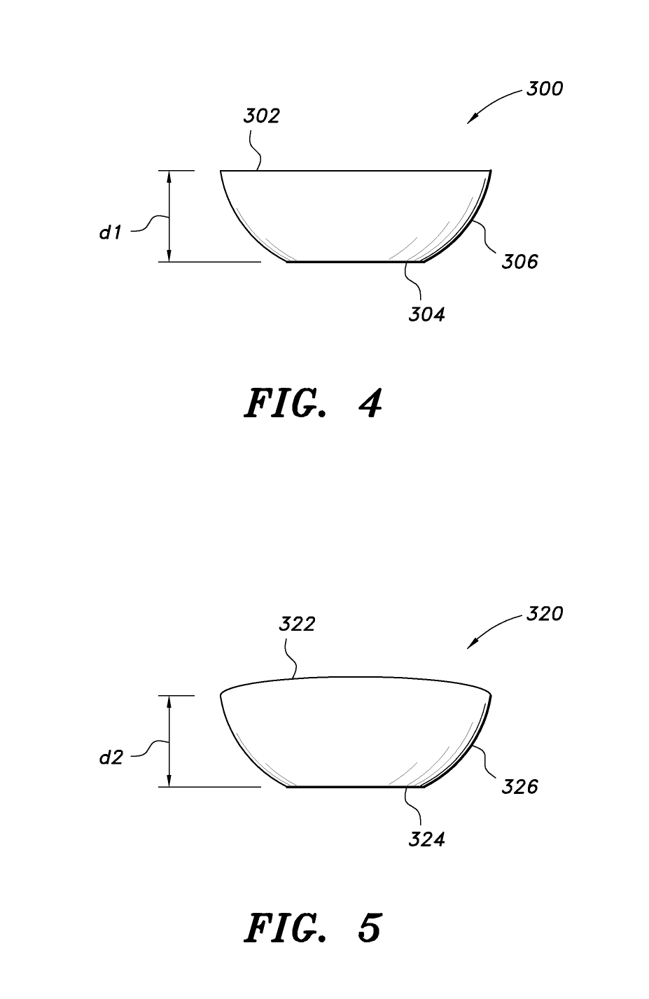

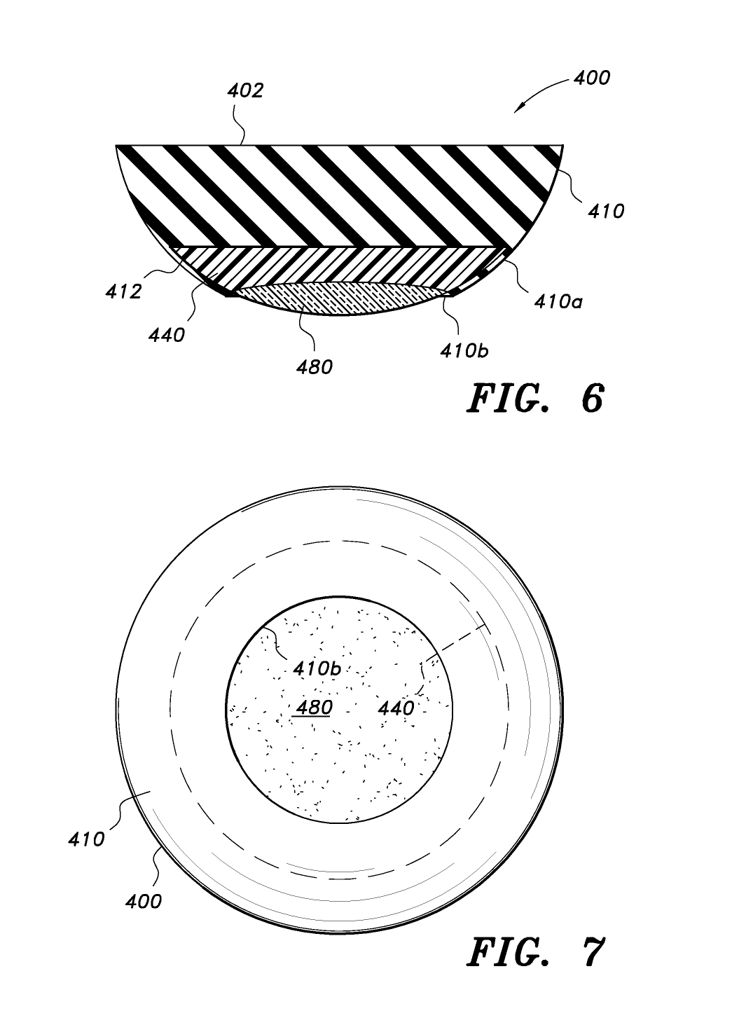

FIG. 4 is a side elevational view of a truncated dome-shaped portion 300, for use with the embodiment of FIGS. 1-3 and with the embodiment of FIG. 6. The truncated dome-shaped portion 300 is preferably a solid body composed of a relatively firm resilient material such as rubber. The truncated dome-shaped portion 300 has an upper surface 302, a lower surface 304, and a curved sidewall 306. The upper surface can be indented or flat, and the lower surface 304 is preferably indented. When the lower surface 304 is indented, the indented portion can be used to retain an absorbent receptacle as shown in FIG. 6. In a preferred embodiment, the upper surface 302 of the portion 300 is approximately the diameter of a quarter, while the lower surface 304 is approximately the diameter of a nickel.

FIG. 5 is a side elevational view of a truncated dome-shaped portion 320, for use with the embodiment of FIGS. 1-3 and with the embodiment of FIG. 6. The truncated dome-shaped portion 320 is preferably a solid body composed of a relatively firm resilient material such as rubber. The truncated dome-shaped portion 320 has an upper surface 320, a lower surface 324, and a curved sidewall 326. The upper surface 322 is preferably rounded as shown, and the lower surface 324 is preferably indented as shown. When the lower surface 324 is indented, the indented portion can be used to retain an absorbent receptacle as shown in FIG. 6. In a preferred embodiment, the upper surface 322 of the portion 320 is approximately the diameter of a quarter, while the lower surface 324 is approximately the diameter of a nickel.

FIG. 6 is a side sectional view of another embodiment of the invention, having a pressure applying portion 400, which is formed by a portion 410, a receptable portion 440, and an absorbent portion 480. The portion 410 is preferably formed of a material such as rubber, and the portion 410 is preferably formed of a material such as plastic. The absorbent portion 480 is preferably composed of fiber material such as cotton together with a coagulant of any type known to any one having skill in the coagulent arts. The portion 410 has a thin-walled portion 410a terminating in a lip 410b, for partially enclosing and retaining the receptacle portion 440 and the absorbent portion 480.

FIG. 7 is a top elevational view of the embodiment of FIG. 6, according to the present invention. The elements shown therein are as described hereinabove with reference to FIG. 6. The rounded shape of the portion 410 and of the absorbent portion 480 are visible in this figure.

The article of FIG. 6 can be made, for example, by molding it in sections and then assembling the sections using glue or adhesive. Alternatively, the materials can be formed such that the portion 410a extends outwardly and is bent inwardly only after insertion of the receptacle portion 440 and the absorbent portion 480. Other manufacturing methods and techniques can be used, within the ambit of skill of any one skilled in the manufacturing and assembly arts for complex plastic and rubber articles.

The invention being thus described, it will be evident that the same may be varied in many ways. Such variations are not to be regarded as a departure from the spirit and scope of the invention and all such modifications are intended to be included within the scope of the claims.

What is claimed is:

1. A coaguband device for applying direct pressure on a bleeding site of a patient's body, comprising:
an adhesive band for attachment to a region of the patent's body adjacent the bleeding site,
an absorbent portion adapted for direct contact with the bleeding site, and
a dome-shaped component enclosed between said absorbent portion and said adhesive band, whereby the band anchors the absorbent portion on the bleeding site and at the same time applies pressure to the bleeding site, wherein the dome-shaped component focuses the pressure exerted by the band so that the absorbent portion absorbs unclotted blood.

2. The device of claim 1, wherein said absorbent portion additionally includes a coagulant material to aid in coagulation at the bleeding site.
